(19) United States
(12) Patent Application Publication
(10) Pub. No.: US 2026/0306398 A1
(43) Pub. Date: Oct. 8, 2026

(54) HYALURONIC ACID HYDROGELS WITH AGENT TO ENHANCE RESORPTION

(71) Applicant: Boston Scientific Scimed, Inc., Maple Grove, MN (US)

(72) Inventor: Yen-Hao Hsu, Shrewsbury, MA (US)

(73) Assignee: Boston Scientific Scimed, Inc., Maple Grove, MN (US)

(21) Appl. No.: 19/640,571

(22) Filed: Apr. 7, 2026

Related U.S. Application Data
(60) Provisional application No. 63/784,679, filed on Apr. 7, 2025.

(51) Int. Cl. A61K 9/00; A61K 9/16; A61K 31/423; A61K 47/36

(52) U.S. Cl. CPC A61K 9/0024; A61K 9/1652; A61K 31/423; A61K 47/36

(57) ABSTRACT

In various aspects, the present disclosure provides a medical hydrogel that comprises crosslinked hyaluronic acid and a white-blood-cell-stimulating agent that is released from the medical hydrogel upon administration to a subject. In other aspects, the present disclosure pertains to kits that contain such a medical hydrogel in a container and a device for administering the medical hydrogel to the subject. In further aspects, the present disclosure provides methods of treating patients that comprise administering such a medical hydrogel to a subject.

### CROSS-REFERENCE TO RELATED APPLICATIONS

This application claims the benefit of U.S. Provisional Patent Application Serial No. 63/784,679 filed on Apr. 7, 2025, the disclosure of which is incorporated herein by reference.

### FIELD

The present disclosure relates to hyaluronic acid hydrogels that contain white-blood-cell-stimulating agents to enhance resorption and to methods of making and using such hydrogels, among other aspects. The hyaluronic acid hydrogels of the present disclosure are useful in various biomedical applications.

### BACKGROUND

Non-animal stabilized hyaluronic acid (NASHA) is a hyaluronic acid preparation that is produced wholly from non-animal sources and is used to form hydrogels that are suitable for injections and as fillers, with good biocompatibility in several applications. One hyaluronic-acid-based hydrogel, available as Barrigel®, is used to minimize radiation-associated side effects during prostate cancer treatment by creating the space in between the prostate and rectum. Barrigel® is crosslinked through an ether linkage and does not contain a labile hydrolysable segment, which would provide for enhanced resorption rates. Resorption data have been published for 96 patients at 3-months post-injection (18.4% mean resorption), 55 patients at 12-months post-injection (57.2% mean resorption), and 20 patients at 18-months post-injection (73.6% mean resorption). See 510(k) Premarket Notification – K220641. https://www.accessdata.fda.gov/cdrh_docs/pdf22/K220641.pdf

However, this resorption time frame is significantly longer than is desired for many applications. While hyaluronic-acid-based hydrogels may be removed by medical intervention (e.g., physical removal of the hydrogels or exposure to hyaluronidase to catalyze the breakdown of the hydrogels), there is a need in the art for hyaluronic acid hydrogels which have enhanced rates of resorption without intervention subsequent to administration.

### SUMMARY

In various aspects, the present disclosure provides medical hydrogels that comprise crosslinked hyaluronic acid and a white-blood-cell-stimulating agent that is released from the medical hydrogel upon administration to a subject.

In some embodiments, the white-blood-cell-stimulating agent is a histone acetyltransferase inhibitor or granulocyte colony-stimulating factor (G-CSF).

In some embodiments, the white-blood-cell-stimulating agent is a histone acetyltransferase (HAT) inhibitor.

In some embodiments, the white-blood-cell-stimulating agent is a p300/CBP inhibitor.

In some embodiments, the white-blood-cell-stimulating agent is A-485.

In some embodiments, which can be used in conjunction with the above aspects and embodiments, wherein the white-blood-cell-stimulating agent is constituted from 0.1 wt% to 25 wt% of a dry weight of the medical hydrogel.

In some embodiments, which can be used in conjunction with the above aspects and embodiments, the medical hydrogel comprises covalently crosslinked hyaluronic acid.

In some embodiments, which can be used in conjunction with the above aspects and embodiments, the medical hydrogel comprises ionically crosslinked hyaluronic acid.

In some embodiments, which can be used in conjunction with the above aspects and embodiments, the medical hydrogel is in particulate form. In some of these embodiments, medical hydrogel comprises particles that range from 0.1 micron to 950 microns in largest cross-sectional dimension. In some of these embodiments, the medical hydrogel is in the form of an injectable particulate suspension.

In some embodiments, which can be used in conjunction with the above aspects and embodiments, the medical hydrogel is a radiopaque medical hydrogel. In some of these embodiments, the radiopaque medical hydrogel has a radiopacity that is greater than 100 Hounsfield units (HU).

In other aspects, the present disclosure pertains to kits that contain a medical hydrogel in accordance with any of the above aspects and embodiments in a container and a device for administering the medical hydrogel to the subject.

In some embodiments, the kits comprise the medical hydrogels in particulate form, a syringe, and a needle.

In other aspects, the present disclosure provides methods treating patients that comprise administering a medical hydrogel in accordance with any of the above aspects and embodiments to a subject.

In some embodiments, the subject is a human subject.

In some embodiments, which can be used in conjunction with the above aspects and embodiments, the administered medical hydrogel is in the form of a particle suspension that is injected into the subject.

In some embodiments, which can be used in conjunction with the above aspects and embodiments, the medical hydrogel is injected to provide spacing between tissues.

In some embodiments, which can be used in conjunction with the above aspects and embodiments, the medical hydrogel is implanted into the subject.

Potential advantages of the present disclosure include the ability to administer medical hydrogels with faster bioresorption rates and/or increased infection resistance due to increased levels of white blood cells.

### BRIEF DESCRIPTION OF THE DRAWINGS

FIG. 1 schematically illustrates a process of forming a medical hydrogel, in accordance with an embodiment of the present disclosure.

### DETAILED DESCRIPTION

The present disclosure provides medical hydrogels that comprise crosslinked hyaluronic acid and one or more white-blood-cell-stimulating agents, which are released from the medical hydrogels upon administration (e.g., injection, implantation) to a subject (e.g., a mammalian subject, particularly, a human subject).

As used herein, a “hydrogel” is a crosslinked polymer that contains water or can absorb water but does not dissolve when placed in water.

Hyaluronic acid is a polysaccharide, and more particularly, a glycosaminoglycan comprising residues of D-glucuronic acid and N-acetyl-D-glucosamine. Hyaluronic acid may be provided in acid form or in salt form (also referred to as hyaluronate). Hyaluronic acid is typically produced by microorganisms, including the genus Streptococcus, which excretes hyaluronic acid to the outside of the body. Examples of hyaluronic-acid-producing microorganisms of the genus Streptococcus include Streptococcus equi, Streptococcus zooepidemicus, Streptococcus equisimilis, Streptococcus dysgalactiae, Streptococcus pyogenes, and variants thereof. In various embodiments, the hyaluronic acid may have a number average molecular weight ranging from 5 kDa to 20000 kDa, for example, ranging anywhere from 5 kDa to 10 kDa to 20 kDa to 50 kDa to 100 kDa to 200 kDa to 500 kDa to 1000 kDa to 2000 kDa to 5000 kDa to 10000 kDa to 20000 kDa (i.e., ranging between any two of the preceding values). Common counterions for use in hyaluronic acid salts include Group I cations such as sodium and potassium, and Group II cations such as magnesium and calcium, and mixtures of the foregoing, among others.

Hyaluronidase is an enzyme that is naturally produced by the body in small quantities. White blood cells have been reported in the literature to promote hyaluronidase generation in vivo. Tortora GJ, Derrickson B (2013-12-31). Principles of anatomy & physiology (14th ed.). Danvers, MA. In the present disclosure, medical hydrogels are provided that release white-blood-cell-stimulating agents to stimulate the production of white blood cells and in turn increase hyaluronidase production, thereby increasing the rate of resorption of the medical hydrogels, and/or antimicrobial resistance of the medical hydrogels.

White-blood-cell-stimulating agents for use in the present disclosure include histone acetyltransferase (HAT) inhibitors and granulocyte colony-stimulating factor (G-CSF). HAT inhibiters include p300/CBP HAT family inhibitors (p300/CBP inhibitors) such as one or more the following, among others: A-485, CBP30, A-241, GNE-049, GNE-781, SGC-CBP30, I-CBP112, CCS1477, anacardic acid, curcumin, C646, garcinol, Lys-CoA, CPI-637, dCBP-1, DC_CP20, E7386, GNE-207, HBS1, ICG-001, KCN1, MYBMIM, NASTRp, NEO2734, Nicur, OHM1, PRI-724, PU139, and Y08197. Thus, although particular embodiments of the present disclosure are described in conjunction with A-485 as a particular white-blood-cell-stimulating agent, other white-blood-cell-stimulating agents may be employed. In some embodiments, the white-blood-cell-stimulating agents may be present in the medical hydrogel in amounts ranging from 0.01 wt% to 25 wt% of the dry weight of the hydrogels, for example ranging anywhere from 0.01 wt% to 0.025 wt% to 0.05 wt% to 0.1 wt% to 0.25 wt% to 0.5 wt% to 1 wt% to 2.5 wt% to 5 wt% to 10 wt% to 25 wt% of the dry weight of the hydrogels.

In some embodiments of the present disclosure, the hyaluronic acid molecules are covalently crosslinked with one another using a suitable covalent crosslinking agent. Examples of covalent crosslinking agents include multifunctional crosslinking agents having two or more reactive groups, such as divinyl sulfone, crosslinking agents having two or more glycidyl groups (e.g., glycerol diglycidyl ether, 1,4-butanediol diglycidyl ether, ethylene glycol diglycidyl ether, propylene glycol diglycidyl ether, diglycidyl ethers of polyethylene glycol and polypropylene glycol, etc.), crosslinking agents having two or more epoxide groups, crosslinking agents having two or more aldehyde groups, crosslinking agents having two or more isocyanate groups, crosslinking agents having two or more carboxylic acid groups, ester groups and ketone groups, among others.

1,4-Butanediol diglycidyl ether (BDDE) is one example of a covalent crosslinking agent. BDDE can react with hyaluronic acid under strong base conditions to form stable covalent bonds and is a widely used crosslinking agent in the preparation of hyaluronic acid hydrogels. Under alkaline conditions, the epoxy groups of BDDE react preferentially with hydroxyl groups in hyaluronic acid.

In one exemplary embodiment, hyaluronic acid may be added to a 1% NaOH solution to dissolve the hyaluronic acid. A white-blood-cell-stimulating agent may then be added to the hyaluronic acid solution, followed by BDDE. The mixture may then be subjected to a crosslinking reaction for several hours at elevated temperature, for example, for 4 hours at 40°C. In other embodiments, the white-blood-cell-stimulating agent may be combined with the BDDE and the mixture added to the hyaluronic acid solution. In further embodiments, the hydrogel may be formed first, followed by exposure to the white-blood-cell-stimulating agent under conditions such that the white-blood-cell-stimulating agent is loaded into the hydrogel.

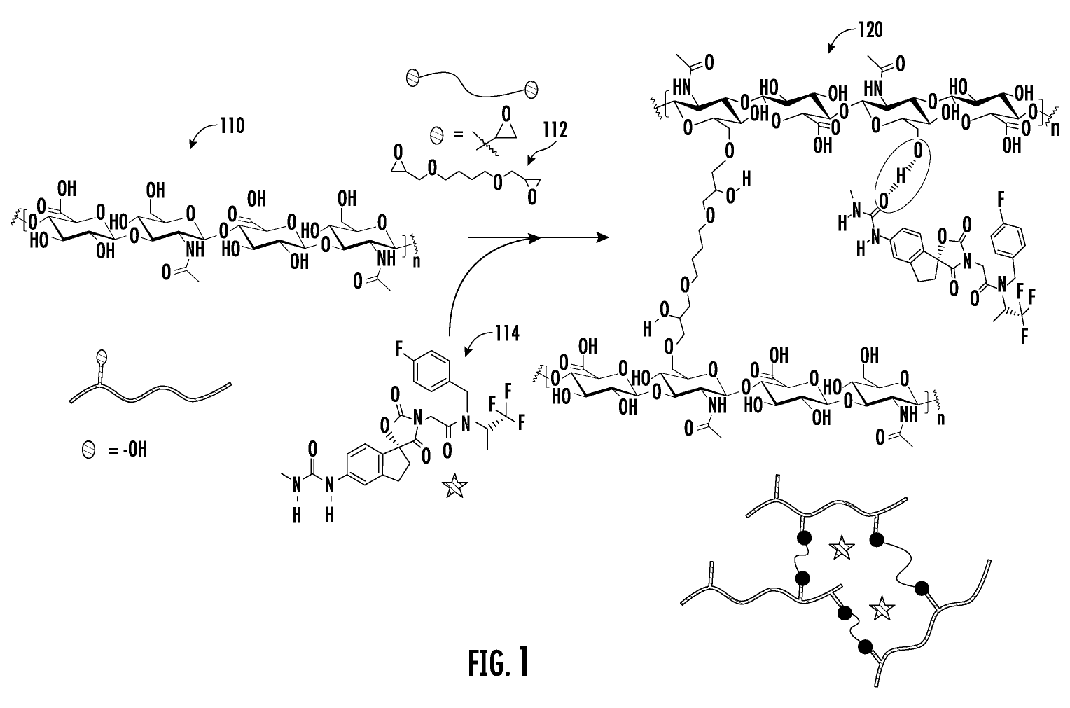

With reference to FIG. 1, in a particular embodiment, hyaluronic acid (110) is shown reacting with 1,4-butanediol diglycidyl ether (112) in the presence of white-blood-cell-stimulating agent A-485 (114) under conditions suitable to form crosslinked hyaluronic acid (120) in which hyaluronic acid molecules are linked together through ether bonds that are formed from reactions between hydroxyl groups of the hyaluronic acid molecules and epoxy groups of the 1,4-butanediol diglycidyl ether. The A-485 (114) is held within the crosslinked hyaluronic acid (120) through intermolecular forces including hydrogen bonding. Although a single hydrogen bond between a carbonyl group of the A-485 and a hydroxyl group of the crosslinked hyaluronic acid is illustrated in FIG. 1, hydrogen bonding between other groups in the A-485 and in the crosslinked hyaluronic acid can occur as well.

In some embodiments, hyaluronic acid molecules are ionically crosslinked with one another using a suitable ionic crosslinking agent. Examples of ionic crosslinking agents include multi-valent cations that can be used to ionically crosslink negatively charged groups of the hyaluronic acid molecules (i.e., in cases where at least a portion of the carboxylic acid groups the hyaluronic acid in the form of anionic carboxylate groups). The negatively charged anionic carboxylate groups of the hyaluronic acid molecules can form ionic bonds (also referred to as electrostatic bonds) with the multivalent cations, which act to ionically crosslink the hyaluronic acid. Examples of multi-valent cations include Mg²⁺, Ca²⁺, Al³⁺, Fe³⁺, Sr²⁺, Ba²⁺, Bi³⁺, Gd³⁺, Ta⁵⁺, W⁶⁺ and Au⁴⁺, among others.

In some embodiments, ionically crosslinked hyaluronic acid may be formed by first deprotonating hyaluronic acid molecules. For example, hyaluronic acid can be treated with a base such as sodium hydroxide or potassium hydroxide to form a hyaluronic acid salt, preferably a monovalent cationic salt, for example, an alkali metal salt such as a lithium (Li⁺), sodium (Na⁺), potassium (K⁺), rubidium (Rb⁺), cesium (Cs⁺) or francium (Fr⁺) salt. Then the monovalent cations are exchanged with multivalent cations, for example, by mixing an aqueous solution of MgCl₂, CaCl₂, AlCl₃, FeCl₃, SrCl₂, BaCl₂, BiCl₃, GdCl₃, TaCl₅, WCl₆, or AuCl₄ with the carboxylate-anion-containing hyaluronic acid forming ionic crosslinks. The A-485 may be, for example, mixed with the hyaluronic acid or hyaluronic acid salt prior to the introduction of the multivalent cations. As another example, the A-485 may be added to the multivalent cations and the resulting mixture added to the hyaluronic acid salt.

In some embodiments, the medical hydrogels of the present disclosure may be radiopaque. Such medical hydrogels have a radiopacity that is greater than 100 Hounsfield units (HU), beneficially anywhere ranging from 100 HU to 250 HU to 500 HU to 750 HU to 1000 HU or more.

In some embodiments, the medical hydrogels contain multivalent cations that are radiopaque. Examples of multi-valent radiopaque cations include Ba²⁺, Bi³⁺, Gd³⁺, Gd³⁺, Ta⁵⁺, W⁶⁺ and Au⁴⁺, among others.

In some embodiments, hyaluronic acid in the medical hydrogels contain may be provided with iodinated side groups. In some embodiments, the iodinated side groups are iodinated aromatic side groups. The iodinated side groups may be covalently linked to the hyaluronic acid through amide or ester bonds as described in US20220204655A1 and US20240335568A1, which are hereby incorporated by reference.

In some embodiments, the medical hydrogels contain radiopaque particles, for example, particles of tantalum, gold, tungsten, rhenium, niobium, molybdenum, and their alloys.

Medical hydrogels in accordance with the present disclosure, may vary widely in size, for example, ranging from 0.1 μm or less to 100 mm or more in largest cross-sectional dimension (e.g., diameter for a sphere, length for a rod, etc.), for example, ranging from 0.1 μm to 0.3 μm to 1 μm to 3 μm to 10 μm to 30 μm to 100 μm to 300 μm to 1 mm to 10 mm to 30 mm to 100 mm.

In some embodiments, the medical hydrogels are in the form of particles. In some embodiments, hyaluronic acid is first crosslinked and then granulated to form crosslinked hyaluronic acid particles of suitable size. Granulating may be by any suitable process, for instance by grinding (including cryogrinding), homogenization, crushing, milling, pounding, forcing the hydrogel through a screen, or the like.

In some embodiments, an emulsion is formed that contains a discontinuous first phase (e.g., an aqueous phase) that contains hyaluronic acid, a white-blood-cell-stimulating agent, and a crosslinking agent that dispersed in a continuous second phase (e.g., an oil phase). Hydrogel particles are formed upon crosslinking of the hyaluronic acid by the crosslinking agent. A homogenizer or sonicator may be used to reduce the dispersed phase droplet size in order to reduce the final crosslinked hyaluronic acid particle size.

Sieving or other known techniques can be used to classify and fractionate crosslinked hyaluronic acid particles according to particle size.

Medical hydrogels in accordance with the present disclosure may contain additional agents. Examples of additional agents include therapeutic agents. Examples of therapeutic agents include antithrombotic agents, anticoagulant agents, antiplatelet agents, thrombolytic agents, antiproliferative agents, anti-inflammatory agents, hyperplasia inhibiting agents, antibiotics, antimicrobials, analgesics, anesthetics, growth factor inhibitors, cell adhesion inhibitors, cell adhesion promoters, anti-angiogenic agents, cytotoxic agents, chemotherapeutic agents, checkpoint inhibitors, immune modulatory cytokines, T-cell agonists, and STING (stimulator of interferon genes) agonists among others.

Examples of additional agents also include colorants such as brilliant blue (e.g., Brilliant Blue FCF, also known as FD&C Blue 1), indigo carmine (also known as FD&C Blue 2), indigo carmine lake, FD&C Blue 1 lake, and methylene blue (also known as methylthioninium chloride), among others.

Examples of additional agents also include imaging agents. Such imaging agents include (a) fluorescent dyes such as fluorescein, indocyanine green, or fluorescent proteins (e.g. green, blue, cyan fluorescent proteins), (b) contrast agents for use in conjunction with magnetic resonance imaging (MRI), including contrast agents that contain elements that form paramagnetic ions, such as Gd^{(III)}, Mn^{(II)}, Fe^{(III)} and compounds (including chelates) containing the same, such as gadolinium ion chelated with diethylenetriaminepentaacetic acid, (c) contrast agents for use in conjunction with ultrasound imaging, including organic and inorganic echogenic particles (i.e., particles that result in an increase in the reflected ultrasonic energy) or organic and inorganic echolucent particles (i.e., particles that result in a decrease in the reflected ultrasonic energy), (d) radiocontrast agents, such as those based on the clinically important isotope ^{99m}Tc, as well as other gamma emitters such as ¹²³I, ¹²⁵I, ¹³¹I, ¹¹¹In, ⁵⁷Co, ¹⁵³Sm, ¹³³Xe, ⁵¹Cr, ^{81m}Kr, ²⁰¹Tl, ⁶⁷Ga, and ⁷⁵Se, among others, (e) positron emitters, such as ¹⁸F, ¹¹C, ¹³N, ¹⁵O, and ⁶⁸Ga, among others, may be employed to yield functionalized radiotracer coatings, (f) radiocontrast agents including those mentioned above as well as non-ionic radiocontrast agents, such as iohexol, iodixanol, ioversol, iopamidol, ioxilan, or iopromide, ionic radiocontrast agents such as diatrizoate, iothalamate, metrizoate, or ioxaglate, and iodinated oils, including ethiodized poppyseed oil (available as Lipiodol®), and (g) contrast agents for use in connection with near-infrared (NIR) imaging, which can be selected to impart near-infrared fluorescence, allowing for deep tissue imaging, for instance, NIR-sensitive nanoparticles such as gold nanoshells, carbon nanotubes (e.g., nanotubes derivatized with hydroxyl or carboxyl groups, for instance, partially oxidized carbon nanotubes), dye-containing nanoparticles, such as dye-doped nanofibers and dye-encapsulating nanoparticles, and semiconductor quantum dots, NIR-sensitive dyes including cyanine dyes, squaraines, phthalocyanines, porphyrin derivatives and borondipyrromethane (BODIPY) analogs, among others.

Medical hydrogels in accordance with the present disclosure may be provided in a fluid form that is suitable for injecting the medical hydrogels or for applying the medical hydrogels onto tissue (e.g., by spraying, extruding, brushing, etc.). Medical hydrogels in accordance with the present disclosure may also be provided in a dry form (e.g., powder form) to which a suitable liquid such as water for injection, normal saline, or phosphate buffered saline may be added to form a fluid medical hydrogel suitable for delivery to a subject. Medical hydrogels in accordance with the present disclosure may be provided in a suitable reservoir, for example, a syringe, vial, or other reservoir.

In various embodiments, kits are provided that include one or more delivery devices for delivering the medical hydrogels to a subject. Such systems may include one or more of the following in any combination: a syringe barrel, which may or may not contain a medical hydrogel as described herein (e.g., in the form of dry particles or a particle suspension); a vial, which may or may not contain a medical hydrogel as described here(e.g., in the form of dry particles or a particle suspension); a needle; a dispenser (e.g., a sprayer); a flexible tube (e.g., adapted to fluidly connect the needle and/or dispenser to the syringe), an injectable liquid such as water for injection, normal saline or phosphate buffered saline. Whether supplied in a syringe, vial, or other reservoir, the medical hydrogels may be provided in dry form (e.g., powder form) or in a form that is ready for injection (e.g., in the form of a hydrogel particle suspension).

The medical hydrogels described herein can be used for a number of medical purposes.

For example, medical hydrogels can be injected to provide spacing between tissues, medical hydrogels can be injected (e.g., in the form of blebs) to provide fiducial markers, medical hydrogels can be injected for tissue augmentation or regeneration, medical hydrogels can be injected as a filler or replacement for soft tissue, medical hydrogels can be injected to provide mechanical support for compromised tissue, medical hydrogels be injected as a scaffold, and/or medical hydrogels can be injected as a carrier of therapeutic agents in the treatment of diseases and cancers and the repair and regeneration of tissue, among other uses. In other examples, medical hydrogels can be ingested to provide gastrointestinal imaging.

After administration, the compositions of the present disclosure can be imaged using a suitable imaging technique. Imaging techniques include ultrasound, x-ray-based imaging technique, such as computerized tomography or X-ray fluoroscopy, and magnetic resonance imaging (MRI).

As seen from the above, the medical hydrogels of the present disclosure may be used in a variety of medical procedures, including the following, among others: a procedure to implant a fiducial marker comprising a medical hydrogel, a procedure to implant a tissue regeneration scaffold comprising a medical hydrogel, a procedure to implant a tissue support comprising a medical hydrogel, a procedure to implant a tissue bulking agent comprising a medical hydrogel, a procedure to implant a therapeutic-agent-containing depot comprising a medical hydrogel, a tissue augmentation procedure comprising implanting a medical hydrogel, a procedure to introduce a medical hydrogel between a first tissue and a second tissue to space the first tissue from the second tissue, or a procedure for diagnostic imaging purposes.

The medical hydrogels may be injected in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra‐vesical injection for urinary incontinence, uterine cavity injection for Asherman's syndrome, submucosal injection for anal incontinence, percutaneous injection for heart failure, intra‐myocardial injection for heart failure and dilated cardiomyopathy, trans‐endocardial injection for myocardial infarction, intra‐articular injection for osteoarthritis, spinal injection for spinal fusion, and spine, oral‐maxillofacial and orthopedic trauma surgeries, spinal injection for posterolateral lumbar spinal fusion, intradiscal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, kidney injection for type 2 diabetes and chronic kidney disease, renal cortex injection for chronic kidney disease from congenital anomalies of kidney and urinary tract, intravitreal injection for neovascular age‐related macular degeneration, intra‐tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production, or ingestion to aid in diagnostic imaging.

As previously indicated, medical hydrogels in accordance with the present disclosure include compositions for therapeutic agent release (e.g., by including one or more therapeutic agents in a matrix of the crosslinked hydrogel) and implants (e.g., compositions for use as tissue markers, compositions that act as spacers to reduce side effects of off-target radiation therapy, cosmetic compositions, etc.), as well as lubricious compositions for medical applications.

1. A medical hydrogel comprising crosslinked hyaluronic acid and a white-blood-cell-stimulating agent that is released from the medical hydrogel upon administration to a subject.

2. The medical hydrogel of claim 1, wherein the white-blood-cell-stimulating agent is a histone acetyltransferase inhibitor or granulocyte colony-stimulating factor (G-CSF).

3. The medical hydrogel of claim 1, wherein the white-blood-cell-stimulating agent is a histone acetyltransferase (HAT) inhibitor.

4. The medical hydrogel of claim 1, wherein the white-blood-cell-stimulating agent is a p300/CBP inhibitor.

5. The medical hydrogel of claim 1, wherein the white-blood-cell-stimulating agent is A-485.

6. The medical hydrogel of claim 1, wherein the white-blood-cell-stimulating agent is constituted from 0.1 wt% to 25 wt% of a dry weight of the medical hydrogel.

7. The medical hydrogel of claim 1, wherein the medical hydrogel comprises covalently crosslinked hyaluronic acid.

8. The medical hydrogel of claim 1, wherein the medical hydrogel comprises ionically crosslinked hyaluronic acid.

9. The medical hydrogel of claim 1, wherein the medical hydrogel is in particulate form.

10. The medical hydrogel of claim 9, wherein the medical hydrogel comprises particles that range from 0.1 micron to 950 microns in largest cross-sectional dimension.

11. The medical hydrogel of claim 9, wherein the medical hydrogel is in the form of an injectable particulate suspension.

12. The medical hydrogel of claim 1, wherein the medical hydrogel is a radiopaque medical hydrogel.

13. The medical hydrogel of claim 12, wherein the radiopaque medical hydrogel has a radiopacity that is greater than 100 Hounsfield units (HU).

14. A kit comprising a medical hydrogel comprising crosslinked hyaluronic acid and a white-blood-cell-stimulating agent that is released from the medical hydrogel upon administration to a subject in a container and a device for administering the medical hydrogel to the subject.

15. The kit of claim 14, wherein the kit comprises the medical hydrogel in particulate form, a syringe, and a needle.

16. A method of treating a patient comprising administering a medical hydrogel comprising crosslinked hyaluronic acid and a white-blood-cell-stimulating agent that is released from the medical hydrogel upon administration to a subject to a subject.

17. The method of claim 16, wherein the subject is a human subject.

18. The method of claim 16, wherein the medical hydrogel is in the form of a particle suspension that is injected into the subject.

19. The method of claim 16, wherein the medical hydrogel is injected to provide spacing between tissues.

20. The method of claim 16, wherein the medical hydrogel is implanted into the subject.
